(19) United States
(12) Patent Application Publication
(10) Pub. No.: US 2014/0316504 A1
(43) Pub. Date: Oct. 23, 2014

(54) IMPLANTABLE INDIFFERENT ELECTRODE

(71) Applicant: Peter Osypka, Grenzach-Wyhlen (DE)

(72) Inventor: Peter Osypka, Grenzach-Wyhlen (DE)

(21) Appl. No.: 14/359,839

(22) PCT Filed: Nov. 21, 2012
(86) PCT No.: PCT/DE2012/001108

§ 371 Date: May 21, 2014

(30) Foreign Application Priority Data
Dec. 3, 2011 (DE) 20 2011 108 639.2

(51) Int. Cl. A61N 1/05

(52) U.S. Cl. CPC A61N 1/0563
USPC 607/122

(57) ABSTRACT

The present invention relates to an electrode arrangement, preferably for the temporary Stimulation or defibrillation of the heart, consisting of at least one unipolar electrode (cathode) (6), which is fixed at the myocardium of the heart, and an indifferent electrode (2a, 2b) which is fixed at the inner wall of the thorax via its supply line and is positioned such that a direct contact to the heart is prevented.

The present invention relates to an electrode arrangement, preferably for the temporary stimulation or defibrillation of the heart.

Catheters, tubes or electrodes are often left temporarily in place to facilitate the faster recovery after surgeries, especially after heart surgeries. In so doing, both the atriums as well as the chambers of the heart are stimulated electrically with an external pacemaker. Two unipolar electrodes (cardiac wires) or one bipolar electrode each are required for the stimulation of every heart chamber. If unipolar electrodes are used, the myocardium of the heart is injured in connection with the attachment of every pole. The outer adipose tissue of the heart often makes it difficult to locate a fat-free position required to attach the electrodes. Two insulated supply lines are always required for attaching bipolar electrodes on the heart. This is disadvantageous particularly for the hearts of children, because the weight or mass of the supply lines must be supported by the relatively small heart.

Temporary electrodes attached on the heart after surgery are activated by a battery-operated external pacemaker. In the past, every electrode consisted of one indifferent and one different pole.

The cables often get tangled with the concurrent temporary stimulation of a plurality of heart chambers, thus compromising the reliability of the stimulation, particularly in connection with the dislocation of the poles. Mistakes frequently happen if the outward-leading lines are not clearly colour-coded for each heart chamber.

Even though four-chamber stimulation following heart surgery would be highly advantageous from an electrophysiological point of view, only what is urgently needed is often initiated for the patient because of the complexity associated with the different cables.

Therefore, the object is to provide an electrode arrangement, preferably for the temporary stimulation of the heart, which allows a reduction of the number of supply lines as compared to conventional arrangements.

To solve the object, the individual indifferent poles are combined to one large surface area pole positioned such that direct contact with the heart is prevented.

Depending on the wiring and design of the pacemaker, a shared indifferent electrode or a series of indifferent electrodes can be placed inside the body such that any direct contact with the heart is avoided or the impairment of its function is prevented.

The present invention therefore relates to an electrode arrangement, preferably for the temporary stimulation or defibrillation of the heart, consisting of at least one unipolar electrode (cathode) (6), which is secured on the myocardium of the heart, and an indifferent electrode (2a, 2b), which is secured on the inner wall of the thorax via its supply line and is positioned such that direct contact with the heart is prevented.

### Advantages

Thanks to the arrangement of the combined indifferent electrode having a large surface area, only a single pole, i.e., only one supply line is required for the stimulation of the four heart chambers. This means that only five lines are required for the stimulation of the four heart chambers: one for the indifferent pole and four lines for each of the four different poles of the heart chambers. For the temporary application, the line to the indifferent pole can be inserted through the opening in the body both separately as well as together with the other poles.

Should the permanent implantation of a pacemaker be planned in the future, the indifferent electrode can also be designed as a permanent implant and connected temporarily with an external pacemaker via an adapter.

The indifferent electrode is metallic and preferably made of platinum, Nitinol, MP35N, a biocompatible stainless steel or conductive synthetic material.

As an example, the indifferent electrode can be a coil or provided as metal braiding, metal strand, metal wire, metal ribbon or metal weave.

The indifferent electrode can be embedded in a protective tube made of synthetic material, preferably silicone, said tube having openings toward the outside such as to allow an electrical current to flow between the unipolar electrode (6) and the indifferent electrode through the openings of the tube.

The indifferent electrode can either be provided as a polished metallic coil or the coil can be embedded in a tube made of synthetic material, preferably silicone, said tube having openings (4, 14, 25). The openings are located on the side facing the heart, thus establishing insulation toward the side of the chest.

The indifferent electrode can also consist of metal braiding, which optionally comprises a membrane facing the side of the heart, said membrane preferably being made of silicone and having openings.

The outer shape, arrangement and size of the metallic coil can be selected to match the patient's anatomy. It is important that the indifferent electrode is arranged on the inside of the thoracic wall opposite the heart chambers such that the delivered defibrillator shock is fully effective.

The indifferent electrode can have different shapes. For instance, it can be designed straight, spiral-shaped, disc-shaped, shamrock-shaped, latticed, ribbon-shaped or polygonal or it can have the shape of a tennis racquet.

The indifferent electrode can comprise a membrane made of soft synthetic material on the side facing away from the heart (18, 22, 26). Aside from silicone, suitable membrane materials include polyurethane, PTFE, polyamide, Pebax and other biocompatible materials, which are suitable as long-term implants. This establishes insulation toward the side of the chest.

The supply lines for the indifferent electrode and the supply line for the unipolar electrode are secured for example by way of synthetic parts, preferably moulded silicone parts, secured by means of ligatures. The fixation can also be achieved with surgical adhesive. The line to the indifferent pole can be guided through the opening in the body both separately as well as together with the supply line to the unipolar electrode.

A further advantage of this electrode arrangement is that the electrodes (preferably coils) can be arranged parallel and pulled out easily from the body when the indifferent electrode is removed through the temporary opening in the chest.

### FIGURES

Additional specifics, characteristics and advantages of the invention can be gathered from the descriptive part below, in which the invention is explained in more detail based on the drawings and exemplary embodiments.

In a schematic representation

FIG. 1 shows the field strength distribution of an exemplary arrangement for stimulation of the heart with an indifferent electrode outside the heart

FIG. 2 shows an exemplary view of the feed-through of an indifferent electrode through the thoracic wall

FIG. 3 shows an exemplary large surface area disc-shaped indifferent electrode

FIG. 4 shows a further exemplary indifferent electrode having a large surface area made from metal braiding

FIG. 5 shows yet another exemplary indifferent electrode having a large surface area

FIG. 6 shows one more exemplary indifferent electrode having a large surface area in the shape of a tennis racquet.

### DESCRIPTION OF THE FIGURES

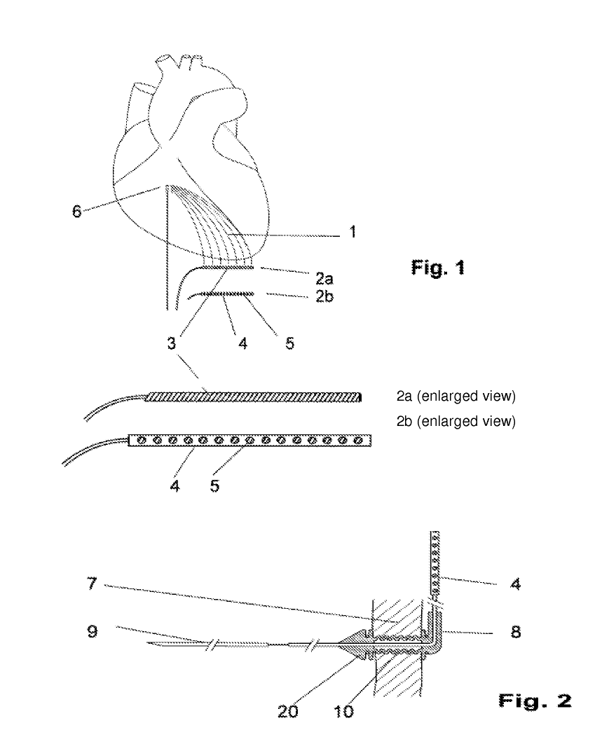

FIG. 1 shows the field strength distribution (1) of an arrangement according to the invention, wherein the indifferent electrode (2a, 2b) is positioned outside the heart. The indifferent electrode is created as metallic polished coil (3) in part (2a). However, the coil can also be embedded in a protective tube made of synthetic material, preferably silicone (4), said tube having openings (5) toward the outside such that an electrical current can flow through the openings between the unipolar electrode (cathode) (6) and the indifferent electrode. The indifferent electrode is positioned such that direct contact of the electrodes with the heart is prevented.

FIG. 2 shows an exemplary fixation of the indifferent electrode through the thoracic wall (7). The lead-through of the indifferent electrode through the thoracic wall (7) can be achieved with a moulded part made of silicone, wherein the moulded silicone part is fixated on the side of the electrode supply line by means of a ligature (20). A chest needle (9) is used to pull the indifferent electrode from the inside to the outside through the thoracic wall. Based on its undulating shape (10), the silicone part (8) can match the respective anatomical conditions of the chest wall thickness. The silicone part (8) can also be combined with the indifferent electrode (4) to form a joint moulded part.

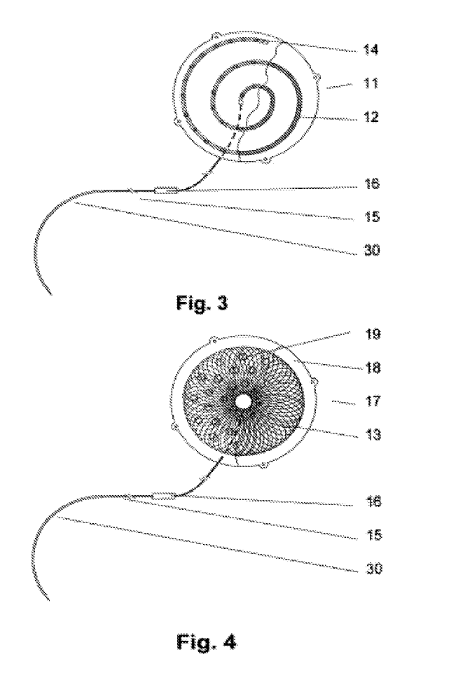

FIG. 3 shows an exemplary disc-shaped indifferent electrode having a large surface area (11). It serves as temporary indifferent electrode for the stimulation of the heart, but can likewise be used as permanent indifferent electrode for implanted pacemakers or implantable cardioverter-defibrillators (ICDs). To prevent direct contact between the coil (12) and the outer wall of the heart, the coil (12) can again—as described in FIG. 1—be integrated into a silicone tube having openings or into a silicone membrane having openings (14). The openings are necessary for the current to flow. A cable (15) connected with a detachable coupling (16), which is pushed through to the outside with a chest needle (30) is used to create the temporary connection with the external pacemaker.

FIG. 4 shows an additional exemplary indifferent electrode having a large surface area (17) made from metal braiding. It is a highly-flexible umbrella braided with individual Nitinol (13) wires. The umbrella is insulated toward the side of the chest with a silicone layer (18) and coated with a perforated silicone membrane (19) toward the side of the heart. A cable (15) connected with a detachable coupling (16) is again used to create the temporary connection with the external pacemaker.

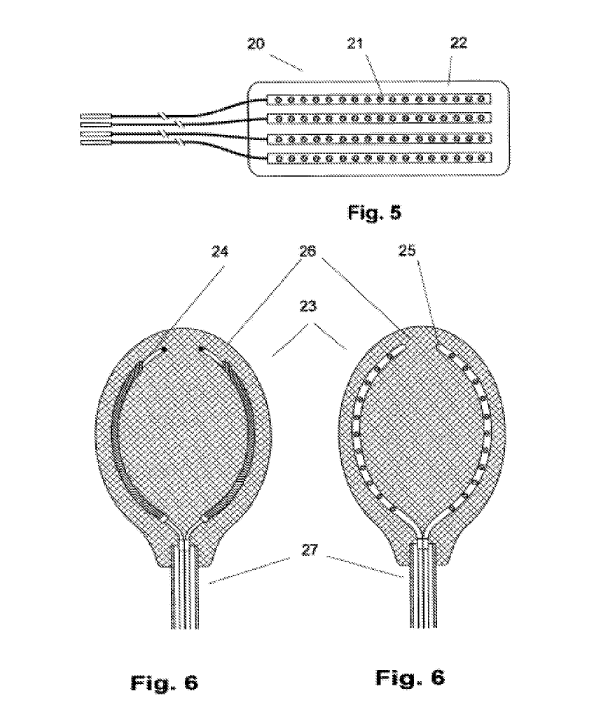

FIG. 5 shows a further exemplary indifferent electrode having a large surface area (20). The perforated silicone sections (21) are designed straight and can be connected as one or a plurality of indifferent electrodes as desired. Optionally, the silicone membrane (22) can be reinforced with fabric made of synthetic material (e.g. Dacron). The electrode is secured on the inside of the chest wall with ligatures, medical adhesive or other common surgical fixation devices. Metallic coils made of platinum, MP35N or other biocompatible conductive materials are used as poles. The supply line for the coil consists of a highly flexible internal metallic strand welded to the coil at the end. Metal braiding, metal weave, metal strand, metal ribbons or metal hemispheres or poles made of conductive synthetic material can be used aside from poles consisting of a coil. As well, the outer shape of the indifferent electrode can have a multiform design depending on the anatomical conditions (circular, rectangular, shamrock-like, latticed, in the shape of ribbons, squares, polygons, etc.). Aside from silicone, suitable membrane materials include polyurethane, PTFE, polyamide, Pebax and other biocompatible materials, which are suitable as long-term implants.

FIG. 6a and FIG. 6b show another exemplary indifferent electrode having a large surface area made of metal coils (24), arranged in the shape of a tennis racquet (23). The coils are insulated toward the side of the chest by a silicone membrane (26) and coated with a perforated silicone tube (25) toward the side of the heart. A cable (27) connected with a detachable coupling (not illustrated here) is again used to create the connection with the external pacemaker.

1. An electrode arrangement, for the temporary stimulation or defibrillation of the heart, comprising: at least one unipolar electrode, which is configured to be secured on the myocardium of the heart, and an indifferent electrode, which is configured to be secured on the inner wall of the thorax via its supply line and be positioned such that direct contact with the heart is prevented.

2. An electrode arrangement according to claim 1, wherein the indifferent electrode is metallic and made of platinum, Nitinol, MP 35N, biocompatible stainless steel or conductive synthetic material.

3. An electrode arrangement according to claim 1, wherein the indifferent electrode is provided as a metal coil, metal braiding, metal strand, metal wire, metal ribbon or metal weave.

4. An electrode arrangement according to any one of claim 1, wherein the indifferent electrode consists of at least one metallic coil.

5. An electrode arrangement according to claim 4, wherein the metallic coil is embedded in a tube made of synthetic material, preferably silicone, said tube having openings, wherein the openings are located on the side facing the heart.

6. An electrode arrangement according to claim 1, wherein the indifferent electrode is made of metal braiding.

7. An electrode arrangement according to claim 6, wherein the metal braiding comprises a membrane having openings on the side facing the heart, said membrane preferably being made of silicone.

8. An electrode arrangement according to claim 1, wherein the indifferent electrode is designed straight, spiral-shaped, disc-shaped, shamrock-shaped, latticed, ribbon-shaped or polygonal or has the shape of a tennis racquet.

9. An electrode arrangement according to claim 1, wherein the indifferent electrode is secured in the patient's thoracic wall by means of a synthetic part.

10. An electrode arrangement according to claim 1, wherein the indifferent electrode comprises a membrane made of soft synthetic material, preferably silicone on the side facing away from the heart.
